(19) United States
(12) Patent Application Publication
(10) Pub. No.: US 2026/0174597 A1
(43) Pub. Date: Jun. 25, 2026

(54) SYSTEM AND METHOD FOR DISINTEGRATING FLOATERS, INCLUDING NEAR-RETINA FLOATERS, USING ADAPTIVE OPTICS

(71) Applicant: Alcon Inc., Fribourg (CH)

(72) Inventor: Zsolt BOR, San Clemente, CA (US)

(21) Appl. No.: 19/415,892

(22) Filed: Dec. 11, 2025

Related U.S. Application Data
(60) Provisional application No. 63/736,257, filed on Dec. 19, 2024.

(51) Int. Cl. A61F 9/008

(52) U.S. Cl. CPC A61F 9/0084; A61F 9/00827; A61F 2009/00851; A61F 2009/00861; A61F 2009/00897

(57) ABSTRACT

A method for treating near-retina floaters includes dilating a pupil of an eye of a patient to at least 6.6 millimeters (mm). While the pupil is dilated to at least 6.6 mm, the method further includes emitting, by an optical delivery system, a plurality of pulses from a treatment laser into the eye of the patient, each pulse of the plurality of pulses having a diameter upon passing through the pupil that substantially fills the pupil and having a focus within 3 mm of a retina of the eye of the patient. Pulses may pass through adaptive optics that may be adjusted based on light emitted from the eye, such as two-photon fluorescence or second harmonic radiation, in order to reduce spot size at the focus.

### CROSS-REFERENCE TO RELATED APPLICATIONS

This application claims the benefit of U.S. Provisional Patent Application No. 63/736,257, filed December 19, 2024, which is incorporated by reference herein in its entirety, and is hereby expressly made a part of this specification.

### BACKGROUND

Light received by the human eye, passes through the transparent cornea covering the iris and pupil of the eye. The light is transmitted through the pupil and is focused by a crystalline lens positioned behind the pupil in a structure called the capsular bag. The light is focused by the lens onto the retina, which includes rods and cones capable of generating nerve impulses in response to the light. The space between the lens and the retina is occupied by a clear gel-like tissue known as the vitreous.

Through various causes, floaters may be present in the vitreous. A floater is typically formed of a clump of cells and collagen fibers or other tissue and is more opaque than the surrounding vitreous. Floaters cast shadows onto the retina that cause visual disturbance for a patient, which can be quite severe in some patients.

It would be an advancement in the art to facilitate the diagnosis and treatment of floaters.

### SUMMARY

The present disclosure relates to a system for disintegrating floaters close to the retina by using feedback control of adaptive optics to reduce the spot size and thereby reduce the pulse energy required to disintegrate the floaters.

In one aspect, a method includes transmitting, from a treatment laser, a treatment beam through adaptive optics and into an eye of a patient, the treatment beam having a numerical aperture such that the treatment beam substantially fills an entire area of a pupil of the eye; detecting, by a detector, two-photon fluorescence or second harmonic radiation from the eye of the patient; and performing, by a computing device, feedback control of the adaptive optics according to an output of the detector to reduce a focus size of the treatment beam within the eye.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates the propagation of light through the eye during femtosecond laser-assisted cataract surgery (FLACS).

FIG. 2 illustrates the propagation of light through the eye during near-retina floater disintegration in accordance with an embodiment of the present invention.

FIG. 3 illustrates a system for performing near-retina floater disintegration in accordance with an embodiment of the present invention.

FIG. 4 is a process flow diagram of a method for performing near-retina floater disintegration in accordance with an embodiment of the present invention.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

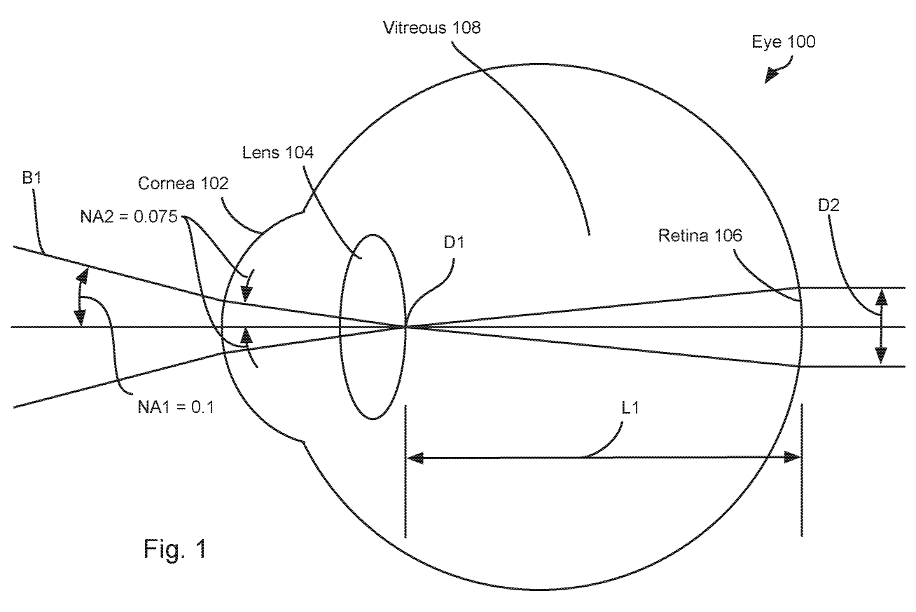

Referring to FIG. 1, the eye 100 includes the cornea 102, which is the transparent outer surface of the eye 100 that receives and focuses light. The light passes from the cornea 102 through the lens 104 and is focused on the retina 106. The vitreous 108 is positioned between the lens 104 and the retina 106.

High-intensity lasers are used in various ophthalmic procedures. For example, femtosecond laser-assisted cataract surgery (FLACS) has been used for many years and millions of procedures. In FLACS, the lens 104 is fragmented in preparation for removal (phacoemulsification) using pulses from a high intensity laser, such as femtosecond laser. Using the approach described in greater detail below, a high-intensity laser may be used to safely treat floaters that are close to the retina 106 (e.g., as close as 1.8 mm from the retina).

In FLACS, the treatment laser beam B1 has a typical numeral aperture of NA1 = 0.1 in air just before the cornea. Due to refraction this numerical aperture is decreased to approximately NA2 = 0.1/1.333 = 0.075 in the anterior and posterior chamber of the eye where 1.333 is the approximate refractive index of the aqueous and vitreous. The numerical aperture is defined as the sine of the half cone angle of a focused beam and is a measure of the strength of the focusing power of the optical delivery system used to deliver the focused beam.

During a FLACS procedure, the beam B1 is focused onto points within the lens with the spot size D1 at the focus being the diffraction limited diameter of the beam B1. Beyond the focal point of the beam B1, the beam B1is incident on the retina 106, which is typically at a distance L1 = 17 mm away from the lens 104, i.e., the typical depth of the posterior segment of the eye 100. The spot diameter D2 of the laser spot on the retina 106 is therefore approximately 0.075*2*17 mm = 2.55 mm. Millions of FLACS procedures have shown that the FLACS procedure is safe and does not cause retinal laser injuries at this 2.55 mm laser spot diameter on the retina.

The numerical values listed above and other numerical values below are provided to facilitate understanding of the approach described herein are not intended to be limiting.

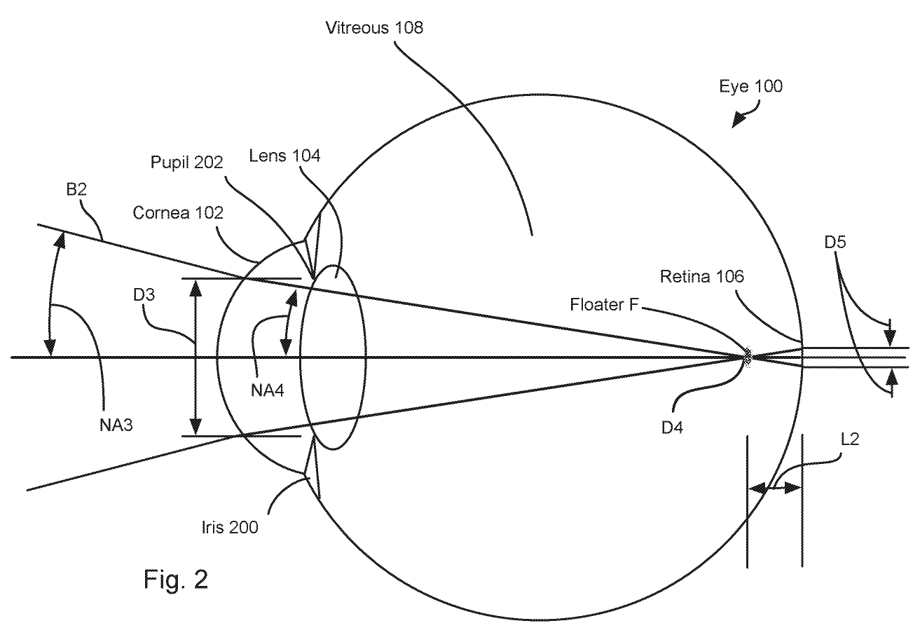

Referring to FIG. 2, floaters F may form at a distance L2 from the retina 106. Many clinically significant floaters, such as the so-called Weiss rings, form at a distance L2 from the retina 106, where L2 is just a few millimeters. For example, the approach described herein may be advantageously used to disintegrate floaters F between 1.8 and 3 mm from the retina 106 that would otherwise be untreatable. Floaters further from the retina may be also be treated in a like manner. Such floaters F are not treatable using conventional approaches due to elevated laser exposure of the retina.

Small laser spot sizes on the retina 106 increase the risk of retinal damage. For example, assume L2 is 2 mm. Further assume a FLACS-like laser beam B1 with an intra-vitreous numerical aperture NA1 = 0.1/1.333=0.075 as for the configuration of FIG. 1. Assuming the focus of the beam B1 is at 2 mm from the retina, the spot diameter on the retina will be 0.075*2*2mm=0.3 mm, which is 2.55/0.3 = 8.5 times smaller than the typical laser spot size during a FLACS procedure, resulting in 8.5^2 = 72.25 more retinal exposure. Such exposure during floater removal can cause unacceptable retinal damage.

FIG. 2 illustrates an approach for treating a near-retinal vitreous floater F using a treatment laser, such as a femtosecond laser. Using the approach of FIG. 2, the iris 200 of the eye 100 is dilated such that the pupil 202 has a diameter D3 of at least 6.6 mm, at least 6.8 mm, or at least 7 mm. The examples below assume a diameter D3 of 7 mm.

A beam B2 from a treatment laser may be used that has a much larger in-air numerical aperture NA3 relative to the beam B1 used for FLACS. The in-air numerical aperture NA3 and numerical aperture NA4 of the beam B2 in the aqueous may be such that the diameter of the beam B2 when passing through the pupil 202 has a diameter of at least 0.8, 0.9, or 0.95 times the diameter D3. The beam B2 is focused on the floater F to a diffraction limited spot size D4 and expands from the focus to a spot size D5 at the retina 106. For example, the beam B2 may have a numerical aperture NA3 of at least 0.280, at least 0.293, or at least 0.307 and a numerical aperture NA4 of at least 0.21, at least 0.22, or at least 0.23.

For example, assume an in-aqueous numerical aperture of NA4 that substantially fills the pupil 202. Further assume the focus of beam B2 is at L2 = 2 mm from the retina 106, e.g., about (17 – 2) mm = 15 mm from the lens 104. This gives a numerical aperture NA4 = ((7 mm / 2) / 15 mm = 0.233, which corresponds to a numerical aperture NA3 = 0.311.

Under these circumstances, the laser spot size D5 at the retina 106 will be 2*NA4*L2 mm = 2*0.233*2 mm = 0.932 mm, which is 2.55/0.932 = 2.736 times smaller than the typical retinal laser spot diameter D2 during a FLACS procedure. This results in 2.726^2 = 7.49 times greater retinal exposure relative to the FLACS procedure. The smaller spot size on the retina 106 is caused by the fact that the floater is 17/2 = 8.8 times nearer to the retina 106 than the lens 104. The in-aqueous numerical aperture NA4 is 0.233/0.075 = 3.1 times larger than the numerical aperture NA2 for the FLACS procedure. As a result, the spot size D4 at the focus is smaller than for the FLACS procedure. The diffraction limited spot size at the focus of a laser beam is inversely proportional to numerical aperture. Therefore, the laser spot size D4 in this example will result in an area of (D2/D4)^2 = (0.233/0.075)^2 = 9.65 times smaller than the area corresponding to laser spot size D2. This 9.65 ratio increases the laser intensity at the focus 9.65 times. Accordingly, the intensity of the laser pulses used to disintegrate the floater F may be correspondingly reduced by up to 9.65 times in order to compensate for the 7.49 times decrease in laser spot area on the retina 106.

In view of the foregoing, assuming compliance with safety standards of a conventional FLACS procedure. The approach illustrated in FIG. 2 may be used to safely treat floaters F near the retina, such as within 3, 2.5, 2.1, or 2 mm of the retina 106. This approach can be used for floaters which are much further from the retina 106.

The above calculations assume that D4 is a diffraction limited laser spot size, i.e. all wavefront errors are removed from the laser beam B2. There are two major sources of the wavefront errors of beam B2. One source is the wavefront errors of the eye caused by the suboptimal anatomical surfaces of the anterior and posterior cornea and of the lens and the spatial inhomogeneities of the refractive index of the lens 104. The other source of the wavefront errors is the laser beam from the laser engine itself and the wavefront errors of the optical delivery system guiding the laser beam from the output of the laser engine onto the cornea. The wavefront errors are typically increasing with the diameter of the beam D3 exponentially therefor the wavefront errors should be removed using adaptive optics.

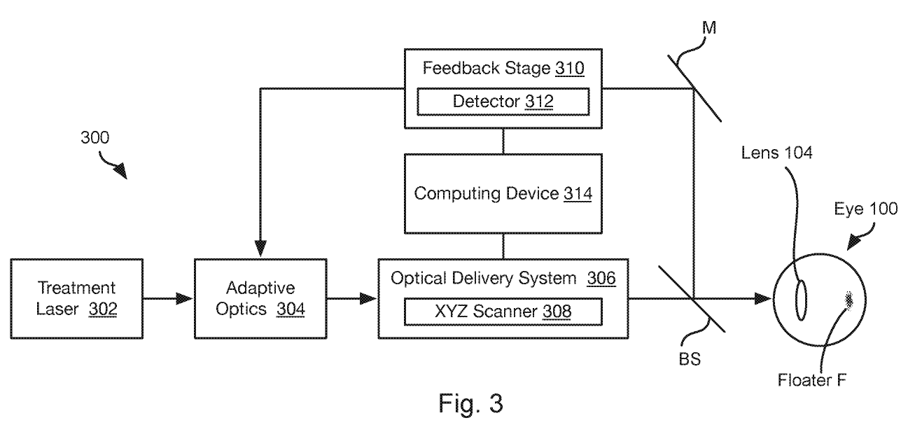

FIG. 3 illustrates a system 300 that may be used to perform laser disintegration of floaters F using laser beam B2 that has been corrected using adaptive optics 304. The system 300 may include a treatment laser 302, such as a femtosecond laser 302. The treatment laser 302 may advantageously have adjustable pulse power to control the laser power properly to take advantage of the decreased spot size D4 at the focus of the beam B2. The treatment laser 302 may include focusing optics or other components of the system 300 may perform any focusing of the treatment laser 302.

The output of the treatment laser engine 302 may be input to adaptive optics 304. The adaptive optics 304 may compensate for optical aberrations caused by the cornea 102 and/or lens 104. For example, the adaptive optics 304 may compensate for coma, trefoil, or other type of high order aberrations. The adaptive optics 304 may or may not provide correction of spherical and astigmatic errors. Such errors can be compensated also by using a prescription contact lens prescribed for the patient by optometrist. The prescription errors can also be pre-compensated by inserting properly selected spherical and a cylindrical lenses into the treatment laser beam, e.g., shifting of the focus of the astigmia-free beam B2. In other embodiments, any spherical adjustment is provided by other components of the system 300. The adaptive optics 304 may be used to reduce the spot size D4 at the focus of the beam B2 in order to increase intensity of the laser beam at the focus.

The output of the adaptive optics 304 may be input to an optical delivery system 306. The optical delivery system 306 includes a scanner 308, such as a three-dimensional (XYZ) scanner 308. The optical delivery system 306 may include one or more scanning mirrors, such as one or more Galvo mirrors, a microelectromechanical system (MEMS) mirror, acousto-optic deflectors, electrically tunable liquid lenses or one or more other types of actuated mirrors. The optical delivery system may include or be embodied as an imaging device, such as an optical coherence tomography (OCT) device, scanning laser ophthalmoscope (SLO), or other type of imaging device. Accordingly, the scanning of a beam from the treatment laser 302 may be performed using the same scanner 308 used by the imaging device. The adaptive optics 304 and/or optics within the optical delivery system 306 may ensure that a focus of the beam from the treatment laser 302 is substantially (e.g., within 1 μm in the XY plane (the plane perpendicular to the optical axis of the eye or within 10 μm in the Z direction parallel to the optical axis) the same as the focus of the imaging device. The output of the optical delivery system 306 may be input to the eye 100 in order to disintegrate a floater.

In some embodiments, feedback may be used to control the adaptive optics 304. For example, a beam splitter BS and possibly one or more mirrors M may direct light reflected or emitted from the eye 100 to a feedback stage 310. The beam splitter BS may be interposed between the optical delivery system 306 and the eye 100 such that a portion of light output from the optical delivery system 306 may pass through the beam splitter BS to enter the eye 100.

The feedback stage 310 may include a detector 312, such as a detector 312 configured to detect intensity of two-photon fluorescence or second harmonic radiation from the focus of the beam B2 located at D4. The maximal intensity of the two-photon fluorescence or second harmonic radiation corresponds to the minimal spot diameter D4 of the focused treatment laser beam, i.e., to the compensated wavefront errors. Accordingly, the higher the output of the detector 312, the smaller the spot size D4 assuming constant pulse energy. The output of the detector 312 may be an electrical signal or digital signal with a magnitude corresponding to the intensity of detected two-photon fluorescence or second harmonic radiation. The feedback stage 310 may use the output of the detector 312 to configure the adaptive optics 304. For example, the feedback stage 310 may implement an optimization or search algorithm configured to search for a combination of parameters defining correction for one or more types of aberration of the eye (astigmatism magnitude and axis, coma, etc.). In particular, the feedback stage 310 may seek to maximize the output of the detector 312. Feedback control of the adaptive optics 304 based on two photon fluorescence or second harmonic radiation of the vitreous can compensate not only the wavefront errors of the dilated eye 100 but also the optical wavefront errors of the treatment laser beam B2 and intrinsic optical errors of the delivery system.

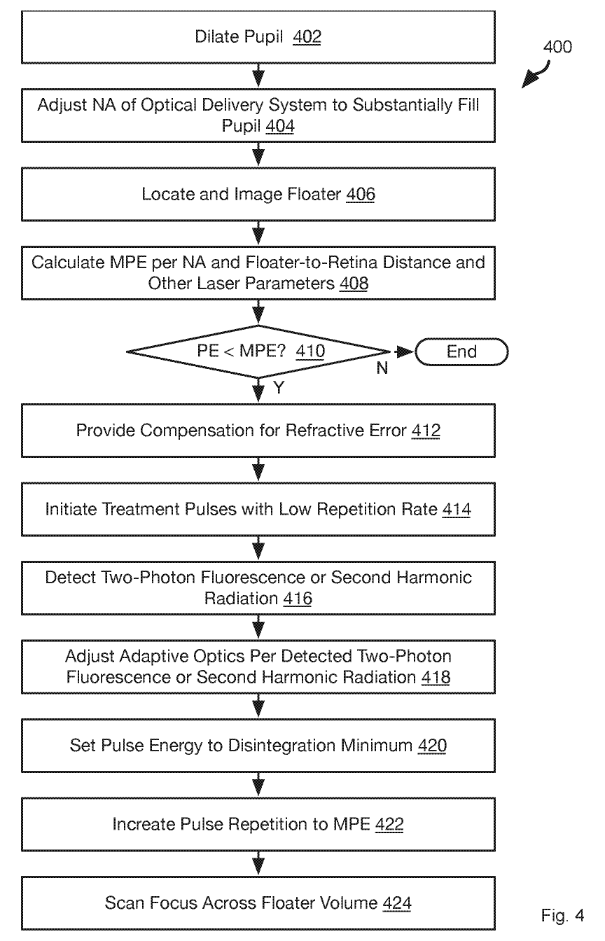

FIG. 4 illustrates a method 400 that may be performed using the system 300 in order to disintegrate a near-retinal floater F. The method 400 may be performed using a computing device incorporated into the feedback stage 310 and/or optical delivery system 306 and/or a separate computing device 314 coupled to the optical delivery system 306 and feedback stage 310.

The method 400 may include dilating, at step 402, the pupil 202 such as using a mydriatic, anticholinergic, or other type of drug causing dilation of the pupil 202. At step 404, the method 400 may include adjusting the NA of the optical delivery system 306 such that a beam (e.g., beam B2) emitted thereby substantially fills the pupil 202 (e.g., at least 80, 90, or 95 percent fills the area of the dilated pupil 202).

The method 400 may include imaging and locating the floater F at step 406. In particular, step 406 may include capturing a volumetric image of the vitreous 108 and identifying points within the vitreous 108 where the floater F is located. Step 406 may be performed manually or automatically, such as using a machine learning model trained to perform this task. Step 404 may include determining vertices of a bounding cube, sphere, ellipsoid, or other shape including the floater F or a shape approximating the contour of the floater F (e.g., a set of vertices).

The method 400 may include calculating, at step 408, a maximum permissible exposure (MPE) that may be emitted by the treatment laser 302 based on the distance of the floater F from the retina 106, e.g., for a point on the floater F closest to the retina 106. For example, the MPE may be calculated based on the spot diameter D5 at the retina 106 assuming a focal point on the floater (e.g., the point closest to the retina 106) and the NA determined at step 404. The MPE may additionally be calculated based on other laser parameters of the beam B2, such as pulse repetition rate, scanning speed, scanning pattern, or other parameters. The MPE may be a maximum allowable intensity and duration of intensity according to a safety standard, such as the American National Standards Institute (ANSI) standard.

The method 400 may include evaluating, at step 410, whether the pulse energy (PE) required to disintegrate the floater F can be emitted into the eye 100 without exceeding the MPE. For example, step 410, may assume a single pulse having sufficient peak intensity and duration to disintegrate a portion of the floater F at the focus of the beam B2 given the NA determined at step 404 and the spot diameter D4 at the floater F according to geometry of the eye 100 (see FIG. 2). For example, the intensity and duration may be sufficient to disintegrate the vitreous 108. If the PE is not found to be less than or equal to the MPE, then the method 400 may end and treatment of the floater F may be deemed unsafe. If not, then subsequent steps of the method 400 may be performed. If the treatment is deemed unsafe due to proximity of the floater to the retina, the surgeon may advise the patient to postpone the surgery a few month. Within a few months the floater typically drift away from the retina and the treatment can be carried out without risking retinal laser damage.

The method 400 may include providing, at step 412, compensation for refractive error of the eye. For example, by placing a prescription contact lens prescribed for the patient by optometrist on the cornea 102 to compensate for spherical error and/or astigmatism. The refractive error can also be pre-compensated by properly inserting properly selected spherical and/or cylindrical lenses into the treatment laser beam B2, e.g., shifting of the focus of the beam B2. Step 412 may also be performed earlier in the method 400, such as before step 404.

The method 400 may include initiating, at step 414, transmitting treatment pulses from the treatment laser 302 into the eye 100 with a low repetition rate, the pulses being focused on one or more points on the floater F. A used herein a “low repetition rate” may be defined as such that each pulse can be considered as a single pulse in terms of potential harm to the retina, e.g., heat buildup is negligible between pulses. Each pulse may be considered a single pulse according to a safety standard, such an ANSI standard.

The method 400 may include detecting, at step 416, two-photon fluorescence or second harmonic radiation resulting from the treatment pulses from step 414, such as using the detector 312. The method 400 may include adjusting, at step 418, the adaptive optics 304, according to the detected two-photon fluorescence or second harmonic radiation.

Steps 414, 416, 418 may be performed repeatedly for multiple pulses in order to tune the adaptive optics 304 and reduce the spot size at the floater F. For example, steps 414, 416, 418 may be performed as part of a search or optimization algorithm in order to determine whether to change parameters defining the operation of the adaptive optics 304 and by how much in order to reduce the spot size at the floater F. The method 400 may include setting, at step 420, the pulse energy of the treatment laser to the minimum required to disintegrate the floater, such as the pulse energy used at step 410. The pulse energy at step 420 may be greater than the pulse energy of pulses output at step 414.

The method 400 may include increasing, at step 422, the pulse repetition frequency setting of the treatment laser 302 to achieve the MPE. In particular, the combination of pulse frequency and pulse energy from step 420 may be set to achieve exposure of the retina 106 that is at or below the MPE, such as within 0.9, 0.95, or 0.99 of the MPE.

The method 400 may include scanning, at step 424, the focus of the treatment laser 302 across the volume including the floater F while emitting pulses from the treatment laser having the pulse energy and pulse repetition rate from steps 420, 422. Step 424 may include performing a raster, spiral, or other scanning pattern across the volume including the floater F. The volume may be the volume determined at step 406.

Step 424 may be performed one or more times until the floater F is disintegrated.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. §112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method comprising:
Transmitting, from a treatment laser, a treatment beam through adaptive optics and into an eye of a patient, the treatment beam having a numerical aperture such that the treatment beam substantially fills an entire area of a pupil of the eye;
detecting, by a detector, at least one of two-photon fluorescence or second harmonic radiation from the eye of the patient; and
performing, by a computing device, feedback control of the adaptive optics according to an output of the detector to reduce a focus size of the treatment beam within the eye.

2. The method of claim 1, further comprising transmitting the treatment beam through an optical delivery system prior to entering the eye of the patient, the optical delivery system placing a focus of the treatment beam on a floater within the eye of the patient.

3. The method of claim 2, further comprising scanning, by the optical delivery system, the focus of the treatment beam over a volume within the eye including the floater.

4. The method of claim 2, wherein the focus of the treatment beam is within 3 millimeters of a retina of the eye.

5. The method of claim 4, wherein the focus of the treatment beam is within 2.1 millimeters of the retina of the eye.

6. The method of claim 2, wherein the optical delivery system includes an imaging device.

7. The method of claim 6, wherein the imaging device is one of an optical coherence tomography (OCT) device or a scanning laser ophthalmoscope.

8. The method of claim 1, further comprising dilating the pupil to at least 6.6 millimeters.

9. The method of claim 1, wherein the adaptive optics are configured to compensate for at least one of astigmatism and coma.

10. The method of claim 1, wherein the treatment laser is a femtosecond laser.

11. A system for treating vitreous floaters, the system comprising:
a treatment laser; and
an optical delivery system configured to deliver pulses from the treatment laser into an eye of a patient, the treatment laser having a numerical aperture of at least 0.28 in air.

12. The system of claim 11, wherein the treatment laser is a femtosecond laser.

13. The system of claim 11, wherein the optical delivery system includes a three-dimensional scanner.

14. The system of claim 13, wherein the optical delivery system includes at least one of an optical coherence tomography (OCT) device or a scanning laser ophthalmoscope.

15. The system of claim 11, further comprising adaptive optics configured to impose at least one of astigmatism and coma correction on an output of the treatment laser.

16. The system of claim 15, further comprising a feedback stage configured to detect emitted light received from the eye of the patient and configure the adaptive optics according to the emitted light.

17. The system of claim 16, wherein the feedback stage is configured to detect at least one of two-photon fluorescence or second harmonic radiation.

18. A method comprising:
dilating a pupil of an eye of a patient to at least 6.6 millimeters (mm); and
while the pupil is dilated to at least 6.6 mm, emitting, by an optical delivery system, a plurality of pulses from a treatment laser into the eye of the patient, each pulse of the plurality of pulses having a diameter upon passing through the pupil that substantially fills the pupil and having a focus on a floater within 3 mm of a retina of the eye of the patient.

19. The method of claim 18, further comprising adaptive optics positioned between the treatment laser and the eye of the patient, the adaptive optics configured to compensate for aberrations of at least one of a cornea or a lens of the eye of the patient, the adaptive optics are configured to compensate for at least one of astigmatism and coma.

20. The method of claim 19, further comprising:
detecting, by a detector, at least one of two-photon fluorescence or second harmonic radiation emitted at the focus; and
selecting, by a feedback stage, parameters defining operation of the adaptive optics according to an output of the detector according to an optimization algorithm seeking to maximize intensity of light emitted at the focus of the plurality of pulses.
